(19) United States
(12) Patent Application Publication
(10) Pub. No.: US 2025/0046335 A1
(43) Pub. Date: Feb. 6, 2025

(54) SPOKEN LANGUAGE UNDERSTANDING BY MEANS OF REPRESENTATIONS LEARNED UNSUPERVISED

(71) Applicant: CORTI APS, Copenhagen K (DK)

(72) Inventor: Lars Maaløe, Copenhagen K (DK)

(73) Assignee: CORTI APS, Copenhagen K (DK)

(21) Appl. No.: 18/713,784

(22) PCT Filed: Nov. 28, 2022
(86) PCT No.: PCT/EP2022/083481

§ 371 Date: May 28, 2024

(30) Foreign Application Priority Data
Nov. 29, 2021 (EP) 21211032.4

(51) Int. Cl. G10L 25/66; G10L 25/30; G16H 10/20

(52) U.S. Cl. CPC G10L 25/66; G10L 25/30; G16H 10/20

(57) ABSTRACT

A method for determining and presenting a recommendation for assisting an interviewee party in a state in need of help solving a problem, such as experiencing a cardiac arrest or an acute injury or disease such as meningitis, during an interview between an inter-viewing party and said interviewee party. The method determines a recommendation as a function of the sound of the interviewee party without an automatic speech recognition routine, i.e. in the processing the sound of the interviewee party is not converted to text (text strings), but instead a feature model is used and the output of that goes direct into a recommendation model.

The present specification relates to a method for assisting a party in a state in need of help solving a problem. The problem may be that the party is experiencing a cardiac arrest or an acute injury or disease such as meningitis.

In case of an emergency situation such as a cardiac arrest, a key component of providing proper care is to summon the emergency medical services (usually an ambulance), by calling for help using the appropriate local emergency telephone number (emergency call). Early recognition that a cardiac arrest has occurred is key to survival—for every minute a patient stays in cardiac arrest, their chances of survival drop.

The party/interviewee party has called the emergency call center and is interviewed by a dispatcher/interviewing party at the emergency call center. The dispatcher may be a voice agent, i.e. a chatbot.

The communication between the interviewing party and the interviewee party may be recorded/captured preferably in real time and routed to a computer system's processing unit where it is processed using a statistical learned model in order to diagnose the problem and determine the most likely cause of action/recommendation.

Another setting may be an interview between an airplane experiencing a problem and an airport tower.

It is contemplated that a computer system may assist in order to arrive at a response recommendation more quickly.

The recommendation may include which treatment is suitable or dispatching emergency medical services such as an ambulance and/or possibly providing a set of pre-arrival instructions to the caller in order for the caller to perform pre-arrival emergency medical service, such as first aid in the case the caller is a bystander to the injured person having the medical emergency. Alternatively, that the injured person is instructed to seek medical assistance at the emergency room or at the local doctor's office. The response action may also be instructions to the cockpit.

A first aspect of the present disclosure is:

A method for determining and presenting a recommendation for assisting an interviewee party in a state in need of help solving a problem, such as experiencing a cardiac arrest or an acute injury or disease such as meningitis, during an interview between an interviewing party and said interviewee party, said method comprising:
providing a sound recorder for capturing the sound of said interviewee party during said interview,
providing a processing unit, and a memory including a database having a feature model comprising a first statistically learned model, said feature model having a first input comprising a number of samples of the sound of said interviewee party, and comprising a first output being a vector or array, said feature model being learned unsupervised,
said database having a recommendation model comprising a second statistically learned model,
said recommendation model having a second input comprising said first output, and a second output comprising said recommendation,
a) inputting a number of samples of the sound of said interviewee party into said feature model and outputting said first output,
b) inputting said first output into said recommendation model and determining said recommendation by means of said recommendation model,
c) determining the confidence level of the output of said recommendation model, and providing a confidence level threshold,
d) when the confidence level being greater than said confidence level threshold: presenting said recommendation to said interviewing party or interviewee party by means of an output device such as a display or a loudspeaker, or
h) when the confidence level being smaller than said confidence level threshold: returning to step a) for inputting a subsequent number of samples of the sound of said interviewee party into said feature model.

A second aspect of the present disclosure is:

A method for determining and presenting a recommendation for assisting an interviewee party in a state in need of help solving a problem, such as experiencing a cardiac arrest or an acute injury or disease such as meningitis, during an interview between an interviewing party and said interviewee party, said method comprising:
providing a sound recorder for capturing the sound of said interviewee party during said interview,
providing a processing unit and a memory including a database having a feature model comprising a first statistically learned model,
said feature model having a first input comprising a number of samples of the sound of said interviewee party, and comprising a first output being a vector or array,
said feature model being learned unsupervised,
said database having a recommendation model comprising a second statistically learned model,
said recommendation model having a second input comprising said first output, and a second output comprising a set of recommendations,
each recommendation in said set of recommendations having a probability of being the true recommendation,
each recommendation in said set of recommendations being a function of said state for solving said problem when presenting a recommendation from said set of recommendations,
a) inputting a number of samples of the sound of said interviewee party into said feature model and outputting said first output,
b) inputting said first output into said recommendation model and determining said plurality of recommendations by means of said recommendation model,
c) determining the respective recommendation in said set of recommendations having the highest, or second highest or third highest probability, and
d) presenting said respective recommendation to said interviewing party or interviewee party by means of an output device such as a display or a loudspeaker.

The processing unit determines the recommendation independently or without interaction from the interviewing party.

The recommendation determined by the processing unit is an expected recommendation, because it is a statistical model that outputs the recommendation, i.e. since it is a statistical model that outputs the recommendation, it is only possible to arrive at a recommendation having a probability of being the true recommendation. But in some cases, there could be a probability of perhaps 100% of being the true recommendation.

The method determines a recommendation as a function of the sound of the interviewee party without an automatic speech recognition routine, i.e. in the processing the sound of the interviewee party is not converted to text (text strings), but instead a feature model is used and the output of that goes direct into a recommendation model.

With the true recommendation is meant the recommendation that in the best way would help solving the problem.

A set of recommendations is to be understood as a plurality of recommendations each being an attempt to solve the problem.

The recommendation model may output another set of output that for example may be related to the facts of the situation, for example that it is a phone call. That the interviewee party is a female or male for example. Such outputs are facts of the situation, but do not solve the problem. A fact of the situation could be output by the processing unit with a higher probability than a recommendation.

The term assisting in the present context means that the interviewing party or interviewee party receives information from the system in order to help deciding on a recommendation, which the interviewing party or interviewee party may or may not use or take advantage of during or after the interview.

A statistically learned model may be a hidden Markov model or a neural network (artificial neural network), and it may be trained with training data in order to find a predictive function, which associates an input and an output.

The training data are samples from the probability distribution according to which the input and output are distributed. The statistically learned model is trained such that the predictive function associates the input and output with the smallest error as possible, i.e. a loss function is defined and minimised.

The interviewing party may be an emergency dispatcher or an air traffic controller or a medical professional (doctor/nurse).

The interviewee party may be a pilot or a bystander to an accident and may be in need of assistance in order to help anyone injured in the accident.

The interviewing party may ask a number of questions to the interviewee party during the interview.

The interview may be a physical interview, or it may be an online interview where both the sound and image of the interviewee party is transmitted electronically.

While the interview takes place, the sound and image of the interviewee party is processed by a processing unit in parallel with the interview—the processing may last the duration of the interview or until a recommendation has been determined, i.e. the processing results in an output that is likely to help with the problem.

During the interview, the processing may result in a first recommendation to one or both of the interview parties, and later in the interview a second recommendation may be presented.

The sound of the interviewee party may be recorded/captured by means of an input device/sound recorder such as a transducer, for example a microphone.

The input device may be connected (wired or wireless) to the processing unit for inputting the sound of the interviewee party into the processing unit as an electronic signal. The electronic signal is a digital signal comprising a sequence of samples (either in the time domain or a domain representative of the frequency contents of the electronic signal).

The sound is sampled either as it is being recorded or following the recording, for example the input device may convert the sound to an analog signal and the analog signal may then be converted to a digital signal. The voice may be sampled in connection with the recording of the voice. The sampling may be at 16 kHz, which is useful for human speech.

The face of the interviewee party (and preferably the background of the interviewee party) may be recorded by means of an input device such as a transducer, for example a camera for capturing a sequence of images of the face of the interviewee party during the interview. Each image may be a digital image.

The images may be captured at a frame rate of for example one image per second. When an interview last two minutes and an image is recorded each second the sequence of images recorded comprises 120 images.

The frame rate may be higher or lower or even adaptable, for example having a higher frame rate in the first part of the interview than in the last part of the interview. The frame rate may also be a function of the sound of the interviewee party, i.e. dependent on the sound of the interviewee party, for example if the sound of the interviewee party comprises an audio feature (such as screaming) indicating that there may be fast varying facial expressions or facial features.

A number of samples (of the electronic signal) at a time may be selected for being processed by (or run through) the feedback model (flowchart symbol 20). The number of samples may correspond to a time interval such as 10 ms of the voice of the interviewee party. Specifically, the number of samples may be constituted by a number of samples between 1 and 300 samples or even up to 1000 samples.

A number of samples are to be understood as a set or collection of samples wherein the samples follow each other chronologically in time. Thus, the number of samples represents a time series or signal, which is an interval or window of the whole interview—the window may be as small as a single sample. Each sample may be represented as an integer or a real number. The processing unit may operate on one sample at a time, i.e. the number of samples being a single sample such that the single sample may constitute the input to the statistically learned model. A sequence of samples is to be understood as samples following each other in time-resulting from the voice of the interviewee party being sampled. A subsequent number of samples is to be understood as a number of samples with the first sample starting at a later point in time than the first sample of the previous number of samples.

The selected number of samples are then input into the processing unit for processing.

When these samples have been processed, the next samples (next time interval with a number of samples) are processed such that a time interval of voice is processed at a time, for example 10 ms sound is processed at a time.

In addition to the samples of sound, a captured image of the interviewee party is selected for being processed, i.e. input into the recommendation model or a facial feature model for determining a signal comprising a facial feature for being input into the recommendation model together with the output from the feature model.

The selected image is then input into the feedback model, such that both samples of sound and an image is processed.

It may be so that a number of time intervals of samples is processed for the same image, i.e. the time interval for a number of samples of sound may be smaller than the time between images (frame rate). Above, it is described that 10 ms of samples may be processed at a time—for an image it may be so that one image is captured per second—corresponding to the frame rate mentioned above.

A time interval of samples that has been processed may not necessarily lead to a feedback/recommendation. The same is the case with an image. In that case no recommendation is presented.

Instead, a next number of samples and perhaps a next image may be processed.

The recommendation model may comprise a statistical learned model such as a neural network, i.e. the model is a model that has been trained using training data. The training data may be historic interviews that have been recorded.

The recommendation model is arranged such that covariance or joint variability between an audio feature and a facial feature is part of the recommendation model, i.e. the recommendation model is not set up to have an independent processing of the sound of the interviewee party and the image of the interviewee party. Thus, the output of the recommendation model is a function of the joint variability between an audio feature and a facial feature.

For example, one or mode nodes in the neural network may have a first input derived from the sound of the interviewee party and a second input derived from the image of the interviewee party, and that node may comprise a function that returns an output that is a function of the joint variability of the first input and the second input.

Each time the recommendation model returns an output, the confidence level may be determined, i.e. the output of the recommendation model may have a confidence level—how likely is it that the output of the model represents the true situation. For example, output returned by the recommendation model may be that the input describes a cardiac arrest—thus, if the confidence level is 95% it is 95% likely that it is a heart attack.

The confidence level may be compared to a threshold. If the confidence level is greater than the threshold, there is confidence in the output of the model, for example that it is probable that there is a risk of cardiac arrest (flowchart symbol 36). The threshold may be 50% such as 60% or 70% or 80% or 90% or 95%.

The threshold may vary on the output. For example, if the output/recommendation is that the interviewee party has described a first situation such as a cardiac arrest, the output may be compared to a first threshold. If the output/feedback is that the interviewee party has described a second situation such as a headache, the output may be compared to a second threshold. The first and second thresholds may be different.

If the confidence level is greater than the threshold, the output/feedback may be presented to the interviewing party by means of the output device.

Unless the interviewing party has decided a response action, such as dispatching an ambulance, the processing may continue by selecting a time interval of samples to be input into the processing. An image may also be selected depending on whether or not a number of time intervals has been processed such that a new image is captured. As mentioned, if a time interval of sound samples corresponds to 10 ms, and a frame rate of capturing images is one second, 100 time intervals of sound are processed for each image.

If the confidence level is smaller than the threshold, no output/recommendation (feedback) is presented—instead the output device could output information that the processing is running, and the processing may continue by selecting a time interval of samples to be input into the processing. This is similar to the situation where the confidence level is greater than the threshold, the difference being whether or not output is presented.

The interviewing party may follow a protocol during the interview for a systematized caller interrogation. The protocol may comprise pre-defined questions, i.e. questions that have been defined before the interview, and the questions may be arranged in successive order—the questions are arranged as a sequence with one question following the other—the sequence being determined before the interview.

The recommendation/feedback may be information to the interviewee party such as what is the expected state of the interviewee party or the situation that the interviewee party is describing. It could be a suggested response action such as to dispatch an ambulance or that the interviewee party should consult a doctor or proceed to the emergency room. The feedback could also be instructions to the interviewee party such as how to perform cpr or how to place an injured person.

The recommendation could also be that the order of the questions in the protocol is re-arranged, for example a question down in the list is moved up and asked earlier in the interview than what was otherwise defined by the protocol before the interview started—for example certain cues or background noise may have a correlation or be associated with certain emergency situations, which means that the processing unit may present questions relating to such an emergency situation sooner than the interviewing party may realize he or she should ask questions relating to such an emergency situation.

The memory may comprise an anomaly model for determining if an input intended for the recommendation model or feature model is an anomaly. An anomaly may be explained by a data point that is outside of the probability distribution of the data that the statistical model is learned upon, i.e. that the data point being an unlikely event. The anomaly may be an anomaly in the sound (audio feature) or an anomaly in the image.

The anomaly model may comprise a first model for determining if the sound comprises an anomaly and a second model for determining if an image comprises an anomaly.

The anomaly model may comprise a statistical learned model as disclosed in WO20239910. The statistical learned model may be constructed such that it encodes or transforms the input signal into a latent probability distribution for example by means of a neural network. The latent distribution may be constituted by a lower dimensionality than that of the probability distribution of the input signal. Samples from the latent distribution may then be decoded back into an approximation to the distribution of the input signal through a second statistical model, i.e. a decoder, which may also be in the form of a neural network. The latent distribution may be conditioned on a known prior distribution during the learning procedure, hence an approximation to the true latent distribution is known. The combination of the latent distribution, prior distribution and the approximation to the input distribution can be collected into an approximation to the confidence. When computing this approximation from an input data point, it explains how well this data point is represented in the data set that the statistical models were learned from. If the difference exceeds a certain threshold, the data point is classified as an anomaly.

An anomaly routine/detection is a test of whether or not the respective interval is an outlier/anomaly of the distribution that the statistically learned model has been trained from, i.e. it is tested if the samples used to train the statistically learned model are also representative of the samples that are provided during live execution of the system (the respective number of samples).

If the respective number of samples is an outlier/anomaly, the statistically learned model will not result in a useful output—it can be said that the statistically learned model had then been trained with too few training samples, which are not representative of the number of samples such that a useful output can be expected.

The processing unit may be part of a system, which may include a number of microphones for recording the voice of the interviewing party and/or the interviewee party, i.e. the voice of the interviewing party may also be input into the feedback model. The system may as well comprise the display and/or speaker for presenting the expected response action to the interviewing party.

The system may also incorporate a telecommunications network in the case that the interviewing party and the interviewee party are located at a distance from each other-such as in an air traffic control tower and a cockpit of an airplane.

The telecommunications network may be a telephone or satellite network connecting the communication terminals of the interviewing party and the interviewee party respectively.

Spoken language understanding (SLU) tasks are usually solved by first transcribing an utterance with automatic speech recognition (ASR) and then feeding the output to a text-based model.

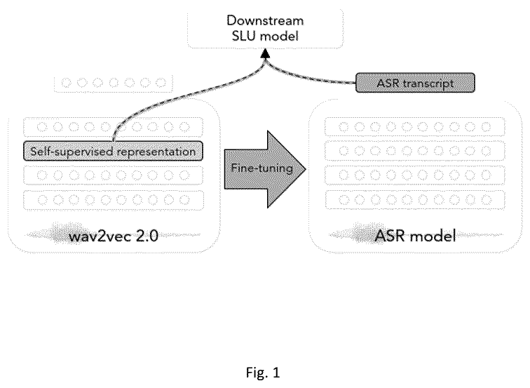

FIG. 1 shows a prior art model such as a self-supervised models like wav2vec 2.0 being fine-tuned for automatic speech recognition.

However, the step of passing the information (signal representing the articulated or spoken sound) through an automatic speech recognition that output text may be redundant for many downstream spoken language understanding tasks where self-supervised representations can be used as input.

Work on speech representation learning has primarily focused on ASR and other speech-specific tasks, such as emotion recognition, speaker identification and phoneme classification. The number of SLU tasks is limited, and many text-based natural language understanding tasks cannot be directly translated to the speech domain due to the difficulty of obtaining word segmentation.

The present disclosure compares speech features from the wav2vec 2.0 model, state-of-the-art ASR transcripts and ground truth text as input for four SLU tasks that all require knowledge about high-level semantic concepts. While the quality of an ASR model can always be debated, the ground truth text defines an upper bound on ASR performance and serves as a baseline. Existing intent classification (IC) and machine translation (MT) tasks are considered and present a speech-based named entity recognition (NER) task.

A proprietary dataset of 911-calls to define a noisy real-world task of cardiac arrest detection (CAD) is used. Our contributions are as follows:

In the following table four tasks, all based on natural speech with dataset statistics are shown.

| | Number of examples | | | Duration | Median | WER |
|---|---|---|---|---|---|---|
| | train | valid | test | [h] | [s] | [%] |
| Named entity | 281,241 | 5,567 | 5,559 | 982 | 16.7 | 2.1/4.5 |
| recognition - | | | | | | |
| LibriSpeech | | | | | | |
| Cardiac arrest | 2,086 | 260 | 260 | 146 | 171.5 | 35.1 |
| detection - Seattle | | | | | | |
| Fire Department | | | | | | |
| Intent classification - | 23,132 | 3,118 | 3,793 | 19 | 1.8 | 23.2 |
| Fluent Speech | | | | | | |
| Commands | | | | | | |
| Machine translation - | 289,165 | 15,517 | 15,524 | 478 | 5.4 | 16.6 |
| CoVoST 2 (En-De) | | | | | | |

The table shows basic dataset statistics for the SLU tasks. Number of examples in the subsets. Duration in hours for all subsets. Median example length in seconds. Word error rate (WER) on the validation set for the ASR models presented.

The named entity recognition LibriSpeech is derived from audiobooks part of the Lib-riVox project1. Training data for wav2vec 2.0 consist of 60K hours of speech from ibriVox, while the open-source ASR models used in this work are trained on LibriSpeech unless stated otherwise. Defining a downstream task on data from the same domain used to train the SSL model and ASR model corresponds to a common scenario here training data for the different modelling steps overlap. LibriSpeech comes with multiple standardized training subsets [9] allowing us to study how the downstream model is affected by varying the amount of training data. Finally, LibriSpeech contains two validation and test subsets, clean and other, which offer insight into the importance of recording quality.

The labels were obtained by using an off-the-shelf Electra language model2 fine-tuned on the CoNLL-2003 NER task, (confer Kevin Clark, Minh-Thang Luong, Quoc V Le, and Christopher D Manning, “Electra: Pre-training text encoders as discriminators rather than generators,” in International Conference on Learning Representations (ICLR), 2019, and Thomas Wolf, Lysandre Debut, Victor Sanh, Julien Chaumond, Clement Delangue, Anthony Moi, Pierric Cistac, Tim Rault, Remi Louf, Morgan Funtowicz, et al., “Huggingface's transformers: State-of-the-art natural language processing,” arXiv:1910.03771, 2019. and applying the model to the ground truth text.

Model-induced labels have been manually reviewed for the validation set to get a sense of data quality. For 1,000 randomly selected examples, human-model agreement is high, with a Krippendorff's alpha of 0.98.

The task is to predict whether a named entity is contained in the input example. In contrast to classic text-based NER, where each word is tagged with a label, we considered thia binary sequence-based classification task to keep the model setup for text and speech features as similar as possible. Conveniently, we find that the dataset is balanced such that approximately 50% of the examples in the training subsets contain a named entity. For validation and test, the fraction is around 30%. We make the dataset available with more details3, such as entity type (i.e., person, location, organization or miscellaneous) and entity alignment obtained from Loren Lugosch, Mirco Ravanelli, Patrick Ignoto, Vikrant Singh Tomar, and Yoshua Bengio, “Speech model pre-training for end-to-end spoken language understanding,” arXiv:1904.03670, 2019. which used the Montreal Forced Aligner4.

From a proprietary dataset of 911 emergency calls provided by Seattle Fire Department, WA, USA, a binary sequence classification task where the objective is to predict whether the caller describes an out-of-hospital cardiac arrest (OHCA) or not is constructed.

The original dataset contains 1303 OHCA calls and many more not-OHCA calls. We did a random 80-10-10 split of the OHCA calls and sampled a not-OHCA call of similar length to each of the OHCA calls to keep the dataset balanced in terms of target distribution and hours of speech per class. We did not have ground truth text available for this task but report word error rate on a separate subset in above table.

The Fluent Speech Commands (FSC) dataset (confer Loren Lugosch, Mirco Ravanelli, Patrick Ignoto, Vikrant Singh Tomar, and Yoshua Bengio, “Speech model pre-training for end-to-end spoken language understanding,” arXiv:1904.03670, 2019) consists of 248 unique read-speech commands from 97 speakers instructing a hypothetical intelligent home device to perform an action (e.g., “Turn the lights on in the kitchen”). Recording of the commands was crowd-sourced, resulting in a varied selection of English speakers from the US and Canada. The task was originally phrased as a multi-slot task with three slots: action, object and location. However, due to the small number of slot classes, the task is commonly rephrased as a simple classification task with 31 unique classes.

CoVoST 2 is a multilingual speech-to-text translation dataset [12] derived from the Common Voice speech corpus [13]. Translations were made by professional translators and the corresponding speech recordings were crowd sourced. We focused on the English-to-German task using the so-called CoVoST training set and the Common Voice test and validation sets as in the original work.

We are interested in comparing the information content of the input representations, so we chose a minimalist architecture. All models take as input a sequence of vectors x1:T=x1, x2, . . . , xT where xt∈RK and share a similar encoder. A fully connected layer without activation maps each xt to a D-dimensional linear subspace. This linear mapping is the only source of variation in terms of model parameterization between the input representations as it depends on the input dimensionality K; 1024 for wav2vec 2.0 representations, 29 for character-level text and 1,296 to 41,341 for word-level text. The linearly projected features are fed to a bidirectional LSTM with a D-dimensional recurrent state. Hereafter, each task requires a different architecture. For the binary NER and CAD tasks, the LSTM output h1:T is max-pooled and fed into a single neuron with a sigmoid activation to parameterize a Bernoulli distribution. Similarly, for the IC task, the LSTM output is pooled and mapped to a 31-dimensional vector with softmax normalization to parameterize a categorical distribution. For the MT task, we used an LSTM-based autoregressive decoder with scaled dot-product attention [14]. We used a vocabulary of 10K subword units for the target language.

The wav2vec 2.0 models use contrastive self-supervised learning and are fine-tuned for ASR with a connectionist temporal classification loss. For more details see Alexei Baevski, Yuhao Zhou, Abdelrahman Mohamed, and Michael Auli, “wav2vec 2.0: A framework for self-supervised learning of speech representations,” Advances in Neural Information Processing Systems (NeurIPS), vol. 33, 2020.

Two SSL-ASR model pairs were downloaded from the FAIRSEQ sequence modeling toolkit6. For the first pair, the self-supervised wav2vec2.0 model has been trained on 60K hours of speech from LibriLight and fine-tuned on 960 hours from LibriSpeech [1]. The second pair, which is more robust, adds 3000 hours of conversational and crowd sourced speech from the Fisher, Switchboard and CommonVoice corpora to the self-supervised training, while the ASR model was finetuned using the 300 hours from Switchboard [15]. All models use the same architecture. We tested the two ASR models on the validation set for each task and chose the model pair corresponding to the lowest word error rate. For the IC and CAD tasks, the robust ASR model was better.

As shown in [6, 16], the top layers of wav2vec 2.0 are a poor choice of input to phoneme classification and ASR. We ran a small initial experiment with limited training data to determine which output from the 24 transformer layers in the wav2vec 2.0 architecture to use as input to the downstream tasks. We found that layer 15 yielded the best results.

This layer has also been found to provide the best results for phoneme classification [6], and layers 13 through 16 have been shown to contain the highest level of correlation with text-based word embeddings [17].

All models were trained by minimizing cross-entropy and use D=256. In the very low-resource settings, we also tested smaller dimensionalities to reduce overfitting, but this did not improve results. We used the Adam optimizer [18] with a fixed learning rate of 3·10-4 for the first half of training be-fore annealing it to 5·10-5 during the second half. Batch size and validation frequency were tuned for each task on the ASR character level. We ensured that the number of training steps was large enough to reach convergence for all experiments.

Results for each of the four tasks are presented below. The metric commonly used in previous work for the existing tasks is used. GT refers to ground truth.

Named entity recognition results on the LibriSpeech test sets. All results are given in F1-scores.

| | | 10 hours | | 100 hours | | 960 hours | |
|---|---|---|---|---|---|---|---|
| Input | Source | Clean | Other | Clean | Other | Clean | Other |
| Characters | GT | N/A | 100 | 15.5 | 80.5 | 91.0 | 92.0 |
| Word | GT | N/A | 100 | 14 | 76.4 | 89.2 | 91.0 |
| Characters | ASR | 84.1 | 99.4 | 11.5 | 79.9 | 90.3 | 88.2 |
| Word | ASR | 82.5 | 98.7 | 10.9 | 75.7 | 88.1 | 88.2 |
| Wav2vec 2.0 | SSL | 84.7 | 99.6 | 6.1 | 83.6 | 90.0 | 86.3 |

Results for cardiac arrest detection, intent classification and machine translation.

| | | CAD | IC | MT |
|---|---|---|---|---|
| Input | Source | F1 score | Accuracy | BLEU |
| Characters | GT | N/A | 100 | 15.5 |
| Word | GT | N/A | 100 | 14 |
| Characters | ASR | 84.1 | 99.4 | 11.5 |
| Word | ASR | 82.5 | 98.7 | 10.9 |
| Wav2vec 2.0 | SSL | 84.7 | 99.6 | 6.1 |

### Named Entity Recognition: LibriSpeech

The wav2vec 2.0 representations showed impressive performance on the 10-hour subset, as seen in table 2, where text-based models were only slightly better than a random baseline. Even with 100 hours of labelled data, they were superior. The gap closed at 960 hours. In general, models trained on ground truth text performed better on the other subset, whereas speech-based models always performed best on the clean subset, highlighting the speech features' sensitivity to noisy conditions. Although the ASR transcripts are also affected by noise, they gave more robust results, as these models performed better on the other subset in all but one case.

On examples that exclusively contain named entities that are out-of-vocabulary, wav2vec 2.0 representations gave an error rate of 23% when trained on 100 hours. ASR transcripts gave a substantially higher error rate of 36%. This underscores the large amount of data needed for robust out-of-vocabulary named entity recognition.

### Cardiac Arrest Detection: Seattle Fire Department

Considering the observation that ASR transcripts are more noise-robust than wav2vec 2.0 representations, we might expect them to fare better on noisy 911-calls. However, as seen in table 3, the wav2vec 2.0 representations still yielded better results. Unlike the NER task, it is possible that speech-based features, such as emotion and rate of speech, might prove useful for this task.

### Intent Classification: Fluent Speech Commands

As mentioned in the task description, every speaker in this dataset read the same 248 commands. As a result, training, validation and test subsets contain the same 248 identical examples when we consider ground truth text, which leads to an accuracy of 100% as seen in table 3. While the task is generally considered to require semantic understanding [3], which is also why we include it here, it can be solved to perfection by a many-to-one sentence recognizer (i.e., different sentences map to the same intent). The wav2vec 2.0 representations were slightly better than the ASR transcripts and very close to the more complex state-of-the-art ASR-based system from [19] which reached 99.7% accuracy.

### Machine Translation: CoVoST 2

The simple ASR-based MT system was a lot worse than ground truth text. The wav2vec 2.0 representations were even worse. These results are not surprising considering the generally large gap between speech and text-based approaches [12]. It is hypothesized that the lack of simple morphological features, like word boundaries, is a challenge to overcome for a shallow model trained on speech-based representations. To test this hypothesis, we trained the model on the ASR character-level transcripts without white-spaces (e.g., HOW ARE YOU→HOWAREYOU) which resulted in a notable drop from BLEU 11.5 to 9.7, but not enough to explain the gap between the two representations.

This disclosure should not be seen as a quest to remove ASR from the SLU pipeline. Automatically generated transcripts offer an important layer of interpretability in modern speech applications. Furthermore, we did not explore how text-based language models can be modified to handle error prone transcripts, which is a promising direction for SLU [2]. However, this work is highly relevant when large quantities of unlabelled speech data can be easily obtained, but no or limited text data is readily available—such as in an emergency call centre.

The disclosure suggests that ASR fine-tuning can be avoided for downstream SLU tasks. Interestingly, it was recently found that word meaning is shifted towards the output layer of the model when wav2vec 2.0 is fine-tuned for ASR [17].

This disclosure highlights the feasibility of extracting this knowledge directly from the pre-trained model.

It is shown that wav2vec 2.0 representations yielded better performance than speech recognition transcripts with up to 100 hours of training data for cardiac arrest detection, named entity recognition and intent classification. Only when 960 hours of labelled training data was available, the speech recognition-based approach yielded a slight improvement on the named entity recognition task. For machine translation, the wav2vec 2.0 representations were inferior to the text-based features. The results on the classification tasks have implications for how to tackle spoken language understanding tasks with limited training data demonstrating that the traditional automatic speech recognition step can be bypassed.
[1] Alexei Baevski, Yuhao Zhou, Abdelrahman Mohamed, and Michael Auli, “wav2vec 2.0: A framework for self-supervised learning of speech representations,” Advances in Neural Information Processing Systems (NeurIPS), vol. 33, 2020.
[2] Cheng-I Lai, Yung-Sung Chuang, Hung-Yi Lee, Shang-Wen Li, and James Glass, “Semi-supervised spoken language understanding via self-supervised speech and language model pretraining,” in ICASSP 2021-2021 IEEE International Conference on Acoustics, Speech and Sig-nal Processing (ICASSP). IEEE, 2021, pp. 7468-7472.
[3] Shu-wen Yang, Po-Han Chi, Yung-Sung Chuang, Cheng-I Jeff Lai, Kushal Lakhotia, Yist Y Lin, Andy T Liu, Jiatong Shi, Xuankai Chang, Guan-Ting Lin, et al., “Superb: Speech processing universal perfor-mance benchmark,” arXiv:2105.01051, 2021.
[4] Santiago Pascual, Mirco Ravanelli, Joan Serra, Antonio Bonafonte, and Yoshua Bengio, “Learning problem-agnostic speech representations from multiple self-supervised tasks,” arXiv:1904.03416, 2019.
[5] Andy T Liu, Shu-wen Yang, Po-Han Chi, Po-chun Hsu, and Hung-yi Lee, “Mockingjay: Unsupervised speech representation learning with deep bidirectional transformer encoders,” in ICASSP 2020-2020 IEEE Inter-national Conference on Acoustics, Speech and Signal Processing (ICASSP). IEEE, 2020, pp. 6419-6423.
[6] Alexei Baevski, Wei-Ning Hsu, Alexis Conneau, and Michael Auli, “Unsupervised speech recognition,” arXiv:2105.11084, 2021.
[7] Loren Lugosch, Mirco Ravanelli, Patrick Ignoto, Vikrant Singh Tomar, and Yoshua Bengio, “Speech model pre-training for end-to-end spoken language understanding,” arXiv:1904.03670, 2019.
[8] Vassil Panayotov, Guoguo Chen, Daniel Povey, and San-jeev Khudanpur, “Librispeech: an ASR corpus based on public domain audio books,” in International Conference on Acoustics, Speech and Signal Processing (ICASSP). IEEE, 2015, pp. 5206-5210.
[9] Jacob Kahn, Morgane Rivi\grave{ }ere, Weiyi Zheng, Evgeny Kharitonov, Qiantong Xu, Pierre-Emmanuel Mazar\acute{ }e, Julien Karadayi, Vitaliy Liptchinsky, Ronan Collobert, Christian Fuegen, et al., “Libri-light: A benchmark for asr with limited or no supervision,” in International Conference on Acoustics, Speech and Signal Processing (ICASSP). IEEE, 2020, pp. 7669-7673.
[10] Kevin Clark, Minh-Thang Luong, Quoc V Le, and Christopher D Manning, “Electra: Pre-training text encoders as discriminators rather than generators,” in International Conference on Learning Representations (ICLR), 2019.
[11] Thomas Wolf, Lysandre Debut, Victor Sanh, Julien Chaumond, Clement Delangue, Anthony Moi, Pierric Cistac, Tim Rault, R\acute{ } emi Louf, Morgan Funtowicz, et al., “Huggingface's transformers: State-of-the-art natural language processing,” arXiv:1910.03771, 2019.
[12] Changhan Wang, Anne Wu, and Juan Pino, “CoVoST 2 and massively multilingual speech-to-text translation,” arXiv:2007.10310, 2020.
[13] Rosana Ardila, Megan Branson, Kelly Davis, Michael Henretty, Michael Kohler, Josh Meyer, Reuben Morais, Lindsay Saunders, Francis M. Tyers, and Gregor Weber, “Common voice: A massively-multilingual speech corpus,” in Proceedings of the 12th Conference on Lan-guage Resources and Evaluation (LREC 2020), 2020, pp. 4211-4215.
[14] Ashish Vaswani, Noam Shazeer, Niki Parmar, Jakob Uszkoreit, Llion Jones, Aidan N Gomez, Lukasz Kaiser, and Illia Polosukhin, “Attention is all you need,” in Advances in Neural Information Processing Systems (NeurIPS), 2017, pp. 5998-6008.
[15] Wei-Ning Hsu, Anuroop Sriram, Alexei Baevski, Ta-tiana Likhomanenko, Qiantong Xu, Vineel Pratap, Jacob Kahn, Ann Lee, Ronan Collobert, Gabriel Synnaeve, et al., “Robust wav2vec 2.0: Analyzing domain shift in self-supervised pre-training,” arXiv:2104.01027, 2021.
[16] Lasse Borgholt, Tycho M S Tax, Jakob D Havtorn, Lars Maaløe, and Christian Igel, “On scaling contrastive representations for low-resource speech recognition,” in ICASSP 2021-2021 IEEE International Conference on Acoustics, Speech and Signal Processing (ICASSP). IEEE, 2021, pp. 3885-3889.
[17] Ankita Pasad, Ju-Chieh Chou, and Karen Livescu, “Layer-wise analysis of a self-supervised speech repre-sentation model,” arXiv:2107.04734, 2021.
[18] Diederik P Kingma and Jimmy Ba, “Adam: A method for stochastic optimization,” in International Confer-ence on Learning Representations (ICLR), 2015.
[19] Yao Qian, Ximo Bianv, Yu Shi, Naoyuki Kanda, Leo Shen, Zhen Xiao, and Michael Zeng, “Speech-language pre-training for end-to-end spoken language understanding,” in ICASSP 2021-2021 IEEE International Conference on Acoustics, Speech and Signal Processing (ICASSP). IEEE, 2021, pp. 7458-7462.

1. A method for determining and presenting a recommendation for assisting an interviewee party in a state in need of help solving a problem, such as experiencing a cardiac arrest or an acute injury or disease such as meningitis, during an interview between an interviewing party and said interviewee party, said method comprising:
providing a sound recorder for capturing the sound of said interviewee party during said interview,
providing a processing unit, and a memory including a database having a feature model comprising a first statistically learned model,
said feature model having a first input comprising a number of samples of the sound of said interviewee party, and comprising a first output being a vector or array,
said feature model being learned unsupervised,
said database having a recommendation model comprising a second statistically learned model,
said recommendation model having a second input comprising said first output, and a second output comprising said recommendation,
a) inputting a number of samples of the sound of said interviewee party into said feature model and outputting said first output,
b) inputting said first output into said recommendation model and determining said recommendation by means of said recommendation model,
c) determining the confidence level of the output of said recommendation model, and providing a confidence level threshold,
d) when the confidence level being greater than said confidence level threshold: presenting said recommendation to said interviewing party or interviewee party by means of an output device such as a display or a loudspeaker, or
h) when the confidence level being smaller than said confidence level threshold: returning to step a) for inputting a subsequent number of samples of the sound of said interviewee party into said feature model.

2. A method for determining and presenting a recommendation for assisting an interviewee party in a state in need of help solving a problem, such as experiencing a cardiac arrest or an acute injury or disease such as meningitis, during an interview between an interviewing party and said interviewee party, said method comprising:
providing a sound recorder for capturing the sound of said interviewee party during said interview,
providing a processing unit, and a memory including a database having a feature model comprising a first statistically learned model,
said feature model having a first input comprising a number of samples of the sound of said interviewee party, and comprising a first output being a vector or array,
said feature model being learned unsupervised,
said database having a recommendation model comprising a second statistically learned model,
said recommendation model having a second input comprising said first output, and a second output comprising a set of recommendations,
each recommendation in said set of recommendations having a probability of being the true recommendation,
each recommendation in said set of recommendations being a function of said state for solving said problem when presenting a recommendation from said set of recommendations,
a) inputting a number of samples of the sound of said interviewee party into said feature model and outputting said first output,
b) inputting said first output into said recommendation model and determining said plurality of recommendations by means of said recommendation model,
c) determining the respective recommendation in said set of recommendations having the highest, or second highest or third highest probability, and
d) presenting said respective recommendation to said interviewing party or interviewee party by means of an output device such as a display or a loudspeaker.

3. The method according to any of the preceding claims, said set of recommendations comprising more than one recommendation

4. The method according to any of the preceding claims, said recommendation model being learned supervised.

5. The method according to any of the preceding claims, said first input comprising a first vector or first array, and said first output comprising a second vector or second array, said second vector or second array smaller than said first vector or first array.

6. The method according to any of the preceding claims, said first input comprising a vector or array having between 6000 and 50000 digits such as 16000 digits or 8000 digits.

7. The method according to any of the preceding claims, said first output comprising a vector or array having between 16 and 1500 digits.

8. The method according to any of the preceding claims, said feature model trained to determine the most coherent signals in the sound of said interviewee party.

9. The method according to any of the preceding claims, said vector or array comprising digits not constituting text nor audio.

10. The method according to any of the preceding claims, said feature model comprising an encoder model.

11. The method according to any of the preceding claims, said feature model comprising a decoder model.

12. The method according to any of the preceding claims, said feature model learned without ground truth supervision.

13. The method according to any of the preceding claims, said subsequent number of samples being subsequent in time to said number of samples.

14. The method according to any of the preceding claims, inputting the sound of said interviewee party into said processing unit as an electronic signal.

15. The method according to any of the preceding claims, separating said electronic signal into a number of samples in the time domain or a domain representative of the frequency contents of said electronic signal.
